(19) United States
(12) Patent Application Publication
(10) Pub. No.: US 2015/0047648 A1
(43) Pub. Date: Feb. 19, 2015

(54) Sterile Drape

(71) Applicant: Medtrak Holding Company, LLC, Wauwatosa, WI (US)

(72) Inventor: Stephen G. Hushek, Wauwatosa, WI (US)

(21) Appl. No.: 14/457,741

(22) Filed: Aug. 12, 2014

Related U.S. Application Data
(60) Provisional application No. 61/865,710, filed on Aug. 14, 2013.
(60) Provisional application No. 61/893,342, filed on Oct. 21, 2013.

(51) Int. Cl. A61B 19/08; B65B 69/00; A61B 19/12

(52) U.S. Cl. CPC A61B 19/08; A61B 19/12; A61B 19/081; B65B 69/00
USPC 128/856; 128/855; 428/411.1; 428/35.2; 428/172; 53/492

(57) ABSTRACT

This disclosure describes a sterile drape which includes an inner material ply and an outer material ply. The inner material ply has a first surface and an opposite facing sterile second surface. The outer material ply has a first sterile surface and an opposite facing second surface, with the first sterile surface of the outer material ply removably coupled to the second sterile surface of the inner material ply. The sterile drape may include a framework coupled to the first material ply and configured to be manipulated to a desired shape. The drape includes multiple material plies in sequential layers, wherein the outermost material plies have a first inner-facing surface and a second opposite outer-facing sterile second surface. The multiple plies can be removed one at a time to expose a new sterile layer if the outer sterile layer is contaminated.

### CROSS-REFERENCE TO RELATED APPLICATIONS

This application is a nonprovisional application which claims the benefit to U.S. Provisional Patent Application Ser. No. 61/865,710, filed Aug. 14, 2013, entitled Sterile Drape and claims the benefit to U.S. Provisional Application Ser. No. 61/893,342, filed Oct. 21, 2013, entitled Multiple Ply Sterile Drape, both patent applications are hereby incorporated herein by this reference in their entirety.

### FIELD OF THE INVENTION

The present disclosure is directed to sterile medical drapes and more particularly to a multiple ply sterile drape.

### BACKGROUND OF THE INVENTION

Surgical drapes are used on a patient during surgical procedures to create and maintain a sterile environment around the surgical site. The surgical drape creates and maintains a barrier that minimizes the passage of microorganisms between non-sterile and sterile areas. To be effective, the drape material must be resistant to blood, aqueous fluid and abrasion as well as being lint free. As a result, the surgical drape prevents blood and other bodily fluids from contaminating the sterile field, i.e., the incision and the patient's skin surrounding the incision.

Various pieces of medical equipment, including large devices such as operating microscopes or C-arm x-ray systems, also need to be draped because they are either in close proximity to a surgical patient or because they are used by sterile personnel. Recently, the range of equipment has expanded to include MRI scanners.

The subject matter discussed in this background of the invention section should not be assumed to be prior art merely as a result of its mention in the background of the invention section. Similarly, a problem mentioned in the background of the invention section or associated with the subject matter of the background of the invention section should not be assumed to have been previously recognized in the prior art. The subject matter in the background of the invention section merely represents different approaches, which in and of themselves may also be inventions.

The apparatus of the present disclosure must also be of construction which is durable and it should also require little or no maintenance to be provided by the user throughout its operating lifetime. In order to enhance the market appeal of the apparatus of the present disclosure, it should also be of inexpensive construction to thereby afford it the broadest possible market. Finally, it is also an objective that all of the aforesaid advantages and objectives be achieved without incurring any substantial relative disadvantage.

### SUMMARY OF THE INVENTION

This disclosure describes a sterile drape which includes an inner material ply and an outer material ply. The inner material ply has a first surface and an opposite facing sterile second surface. The outer material ply has a first sterile surface and an opposite facing second surface, with the first sterile surface of the outer material ply removably coupled to the second sterile surface of the inner material ply. The sterile drape may include a framework coupled to the first material ply and configured to be manipulated to a desired shape.

In another embodiment the drape includes multiple material plies in sequential layers, wherein the outermost material ply has a sterile first inner-facing surface and a second outer-facing surface. Each material ply's inner-facing sterile first surface is removably coupled to the sterile outer-facing second surface of the adjacent material ply. The multiple plies can be removed one at a time to expose a new sterile layer if the outer sterile layer is contaminated.

In another embodiment, the sterile drape includes a tensioning system which includes a drawstring disposed within the sterile drape and approximate an outer edge of one of the first material ply and the second material ply, with the drawstring extending through a one-way ratchet mechanism defined in one of the plies.

The sterile drape is configured in one of a tube, a planar sheet, and a predetermined shape, with the first material ply and the second material ply removably coupled together with one of adhesive, tape, and thread. In another embodiment the sterile drape is configured in a shape that conforms to the object being draped.

In some embodiments, the sterile drape includes a sterile device attached to each of the sterile surfaces of the inner and outer material plies and any ply of a plurality of material plies, with at least one of the material plies configured to release from the sterile device. In another embodiment, the sterile drape includes a wire terminal configured to receive a wire connector on each of the first and second surfaces of one of the first material ply and second material ply and configured to provide a continuous electrical connection through the drape. In another embodiment each material ply in a plurality of material plies has a wire terminal coupled to the wire terminal in the adjacent material ply to provide a continuous electrical connection through the entire drape. In all embodiments disclosed herein the ply material has characteristics of resistance to blood, aqueous fluid, abrasion, and they are lint free. Typically the sterile drape is a single use device.

There is also disclosed a sterile drape that includes a plurality of material plies disposed between the inner material ply and the outer material ply, with each ply of the plurality of material plies including a first sterile surface and an opposite facing second sterile surface. The inner-facing sterile first surface of each material ply of the plurality of material plies is removably coupled to the sterile outer-facing second surface of the adjacent material ply. The multiple plies can be removed one at a time to expose a new sterile layer if the outer sterile layer is contaminated.

In another embodiment the sterile drape includes an internal framework that allows it be configured in a desired shape.

In another embodiment the inner material ply is colored and the outer material ply is transparent.

In another embodiment alternating transparent material plies have different colors

In another embodiment the drape includes gloves integrated into the ply so that an inner ply can be manipulated without contamination of the inner ply. The clear second ply of a drape with 2 plies or the alternating ply colors in a multi-ply drape facilitates differentiation of the plies and facilitates manipulation of the inner ply through the outermost ply.

There is also disclosed a method to provide a sterile field on an object, wherein the object is one of equipment and a patient.

The method to provide a sterile field on an object includes obtaining a sterile drape package and opening the package and removing the sterile drape. The sterile drape is configured in one of a tube and planar sheet and a predetermined shape, for example a shape that conforms to the object being draped or a shape that is beneficial for the operation of the object being draped. The sterile drape includes an inner material ply having a first surface and an opposite facing sterile second surface and an outer material ply having a first sterile surface and an opposite facing second surface. The first sterile surface of the outer material ply is removably coupled to the sterile second surface of the inner material ply, with one of adhesive, tape, and thread so that the two drape plies behave as a single sheet during application The first surface of the inner material ply and the second surface of the outer material ply may be contaminated during the application of the drape.

The sterile drape is then placed on and coupled to an object with the first surface of the inner material ply placed on the object. The drape is unfolded until the selected parts of the object are covered. Then the outer material ply is removed from the inner material ply without contaminating the sterile second surface of the inner material ply, exposing the sterile second surface of the inner material ply and providing a sterile field. Coupling the first material ply to the object being draped prevents accidental removal of the drape from the object when the second material ply is removed from the first material ply.

In another embodiment, the sterile drape includes a tensioning system and the method includes selectively operating the tensioning system to enclose a portion of the object with the sterile drape. The tensioning system includes a drawstring disposed proximate an outer edge of one of the inner material ply and outer material ply, with the drawstring extending through a one-way ratchet mechanism defined in at least one of the plies.

In another embodiment, the sterile drape includes a sterile device attached to each of the sterile surfaces of the inner and outer material ply and using the sterile device after releasing at least one of the inner and outer material ply from the sterile device.

In another embodiment the sterile drape includes multiple material plies and the method includes removing the outermost material ply when it is contaminated, without contaminating the sterile surface of the adjacent material ply, and exposing the sterile surface of the adjacent material ply to present a sterile surface. Each new outermost material ply may be sequentially removed when the outermost material ply is contaminated or when a new sterile surface is required.

The apparatus of the present invention is of a construction which is both durable and long lasting, and which will require little or no maintenance to be provided by the user throughout its operating lifetime. Finally, all of the aforesaid advantages and objectives are achieved without incurring any substantial relative disadvantage.

### DESCRIPTION OF THE DRAWINGS

These and other advantages of the present disclosure are best understood with reference to the drawings, in which:

FIG. 1 is a schematic illustration of a patient and surgical equipment with an exemplary embodiment of a sterile planar portion of a sterile drape on the patient and on the equipment and illustrating additional equipment disposed between two material plies of the sterile drape,

FIG. 2 is a detail end view of the sterile drape illustrated in FIG. 1 along the line 2-2,

FIG. 3 is a schematic illustration of equipment covered with a sterile drape including an exemplary embodiment of a tensioning system configured to secure a material ply of the sterile drape to the equipment,

FIG. 4 is a schematic exploded illustration of an exemplary embodiment of a tube sterile drape having a first and second material ply disclosed herein configured for disposition within a cylindrical magnetic resonance imaging magnet,

FIG. 5 is a partial section view of an exemplary embodiment of a one-way ratchet mechanism of the tensioning system coupled to the sterile drape illustrated in FIG. 3,

FIG. 6 is a side view of a drawstring extending from the one-way ratchet mechanism illustrated in FIG. 5,

FIG. 7 is a detail end view of the sterile drape illustrated in FIG. 1 with a sterile device sandwiched between the two material plies,

FIG. 8 is a schematic illustration of a patient and surgical equipment with an exemplary embodiment of a sterile planar portion of a multiple ply sterile drape on the patient and on the equipment and illustrating additional equipment disposed between material plies of the sterile drape and illustrating multiple plies,

FIG. 9 is a schematic exploded illustration of an exemplary embodiment of a tube-type sterile drape having multiple material plies disclosed herein configured for disposition within a cylindrical magnetic resonance imaging magnet,

FIG. 10 is a schematic illustration of equipment covered with a sterile drape including an exemplary embodiment of a plurality of cavities configured as a glove in a material ply of the sterile drape to facilitate positioning the drape on the equipment and coupling the drape to the equipment illustrating a transparent outer material ply,

FIG. 11 is a detail end view of the sterile drape illustrated in FIG. 8 along the line 11-11,

FIG. 12 is a detail end view of the sterile drape illustrated in FIG. 8 along the line 12-12 illustrating cavities defined for sterile devices sandwiched between the material plies of a multiple ply sterile drape.

### DETAILED DESCRIPTION OF THE EXEMPLARY EMBODIMENTS

Draping equipment can be difficult because of odd shapes of the equipment or large size which can make it difficult to get the drapes over or around the equipment. The necessity to drape the equipment from one end and proceed towards the other to maintain sterility of the draped surface can lead to inconveniently tight or loose sections of the drape. Draping large pieces of equipment often requires cooperation between a sterile team member and a non-sterile team member, further complicating the process. The difficulties can contribute to lengthy times for application of the drapes and contamination or tearing during application. The high cost of operating room time and the interest in minimizing the patient's time under general anesthesia contributes to high value for drape systems that can be more easily or quickly applied.

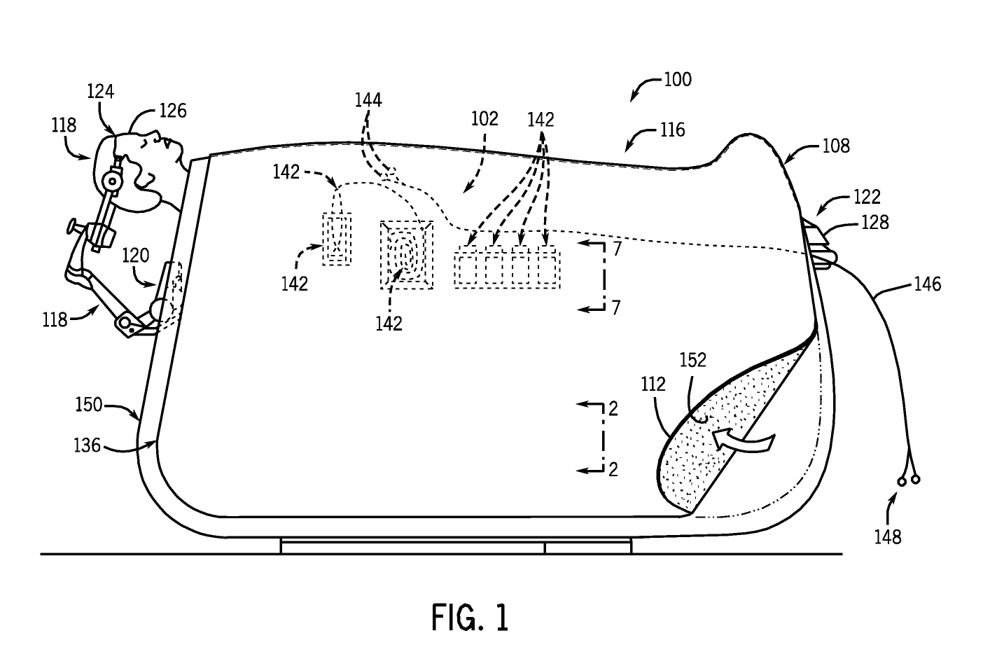

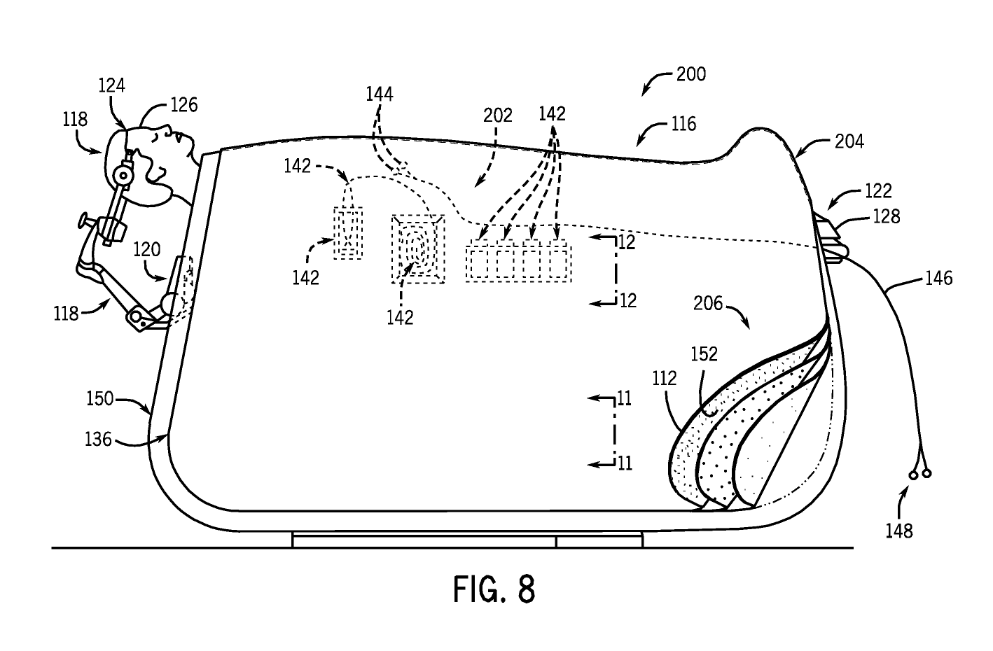

Referring to the FIGS. 1-12, a dual-ply drape (FIG. 1) and a multi-ply drape (FIG. 8) and their application technique disclosed and claimed herein have several advantages. The likelihood of contamination is reduced because the exposed surfaces of the drape 100 do not need to be maintained as sterile during the application. The drape 100 can be applied by non-sterile personnel, allowing the sterile personnel to focus on tasks that specifically require sterile handling. Also, the drapes can be applied in advance without concern for contamination because the second ply does not have to be removed until the object needs to be sterile. This can improve the workflow of the clinical procedure and possibly reduce the amount of time the patient is under general anesthesia because removing the second ply of the drape will be much faster than draping the equipment with the standard techniques requiring sterile personnel.

For purposes of this application, a dual ply drape 100 refers to a drape having two plies or two layers. A multi-ply drape 200 refers to a drape having more than two plies or more than two layers, with the material ply closest to the object 118 referred to as an inner, innermost or first material ply 202 and the ply closest to the person applying the drape referred to as an outer, outermost or second material ply 204

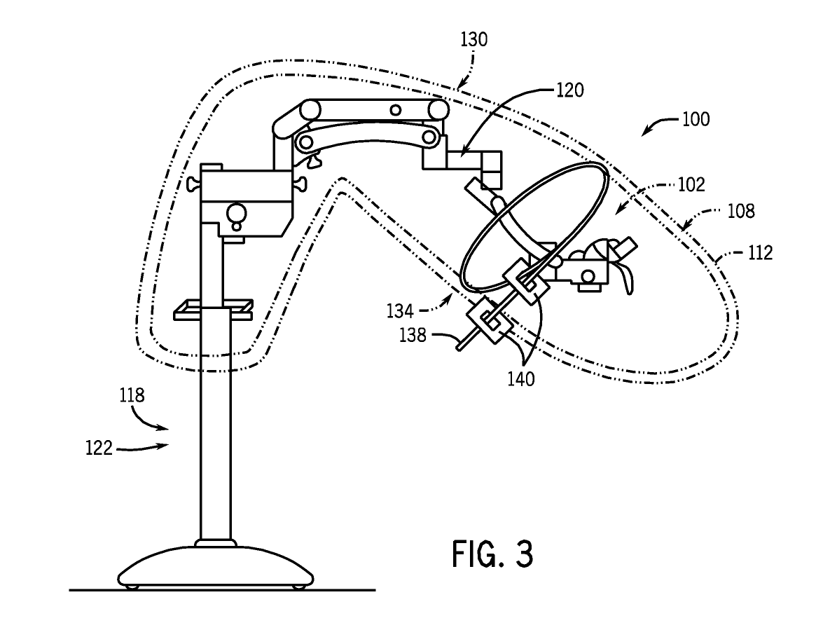

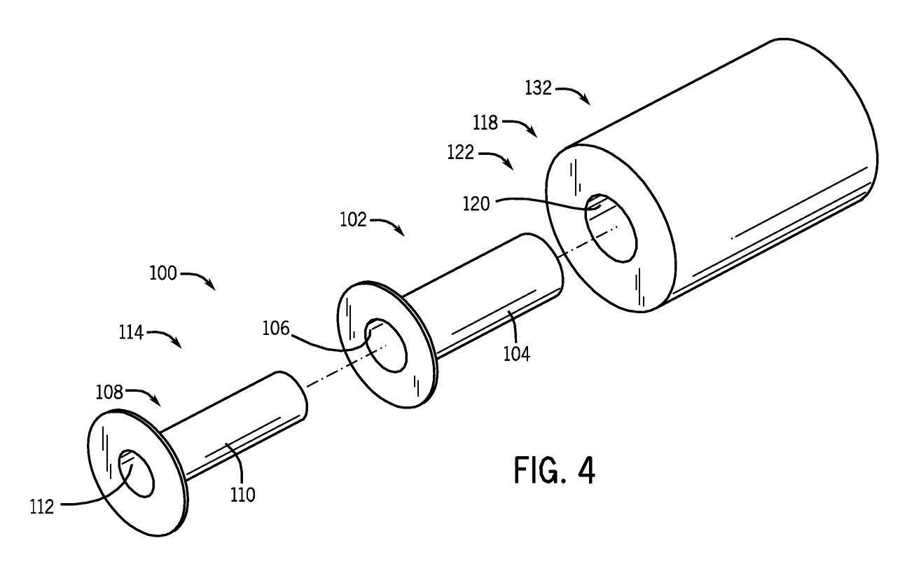

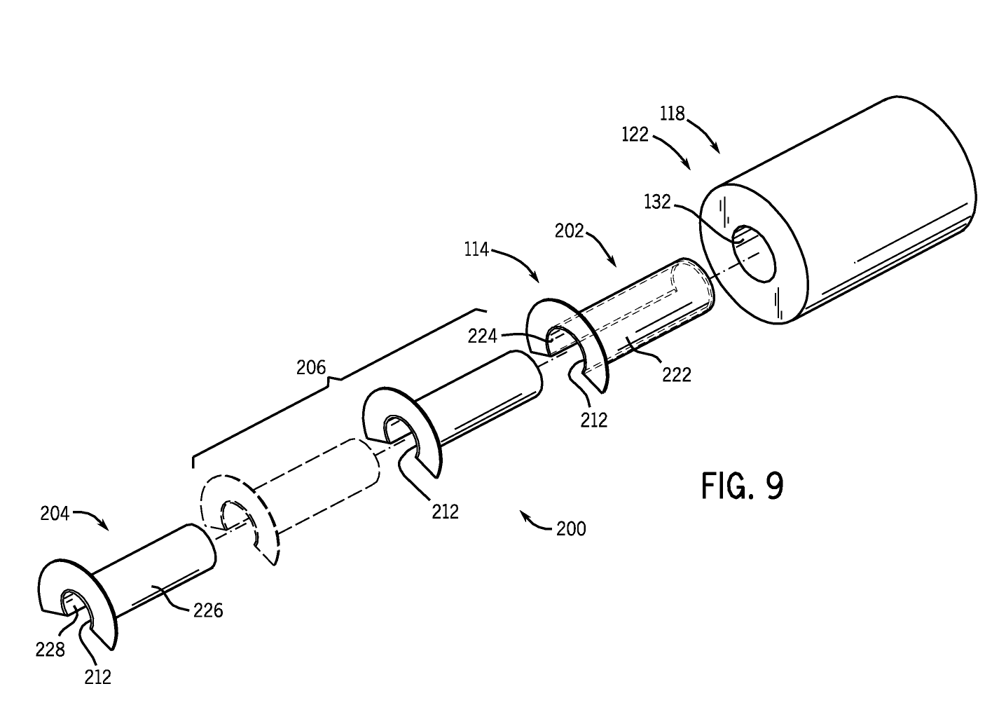

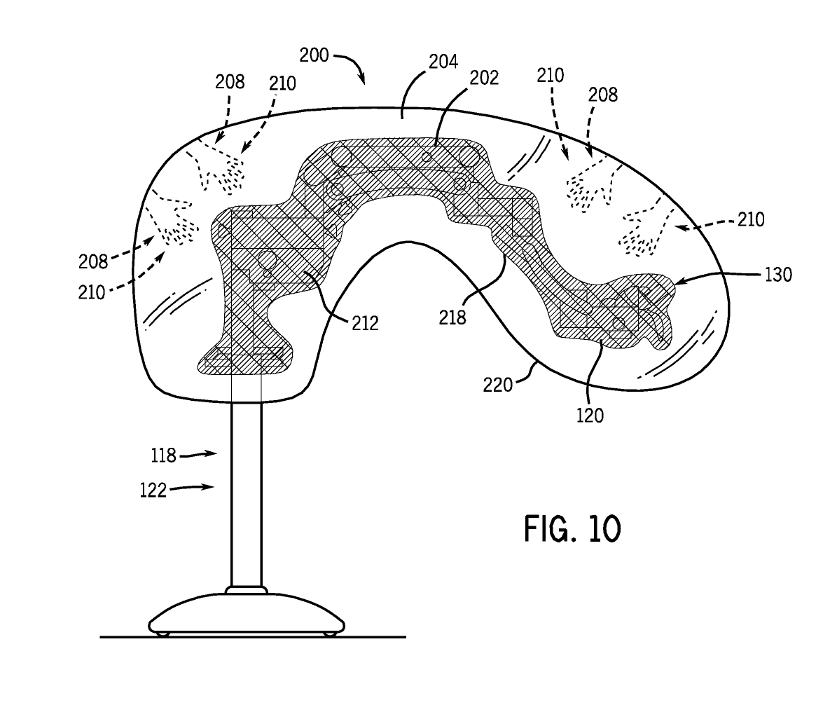

In some situations, large pieces of equipment 122 interfere with the workflow within the operating room, such as a microscope 130 (See FIG. 3), and C-arm x-ray systems. C-arms are large and bulky and the operating rooms are often not large enough for the surgery to continue while the equipment is brought in and draped. The sterile drape 100 or 200 can be configured as a tube 114 (FIGS. 4 and 9) or a planar sheet 116 (FIGS. 1 and 8) or a predetermined shape, for example, a shape that conforms to the object being draped or that is appropriate for the operation of the object being draped (See FIG. 10). A shape of the drape 100 that perfectly conformed to an object may, in some instances, inhibit operation of the object if the object articulates or changes shape, so a different, predetermined non-conformal shape of the drape may be required for advantageous operation (See FIG. 3).

The multi-ply drape 200 disclosed herein can be applied by non-sterile personnel while the C-arm or other equipment is in the hallway, then the second ply removed after the C-arm has been moved into the operating room. Similarly, the bore of a magnetic resonance imager 132 may be draped (See FIGS. 4 and 9) using a sterile drape 100, 200 configured as a tube 114 while the patient 124 is still in the operating room, then the second ply removed just before the patient is moved to the scanner. This allows the surgical staff to observe the removal of the second or outer ply and insure maintenance of the sterile field throughout the procedure.

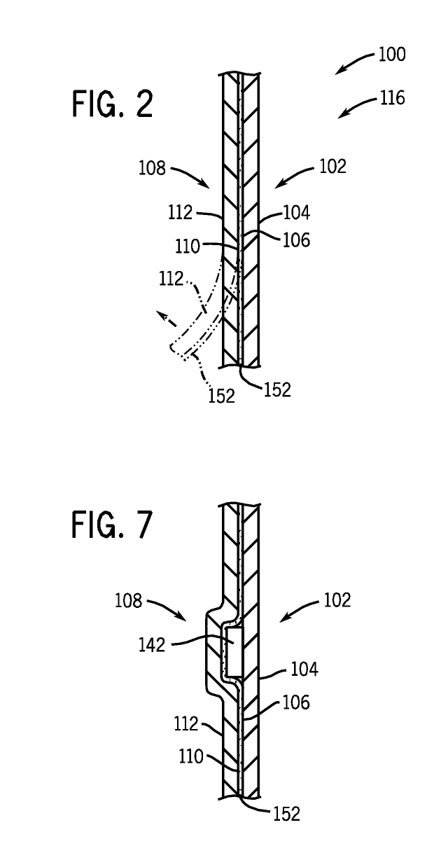

There is provided a double ply drape 100 configured to expedite the drape application. The sterile drape 100 is composed of two plies 102, 108 of drape material. A first material ply 102 has a first surface 104 and an opposite facing sterile second surface 106. The second material ply 108 has a first sterile surface 110 and an opposite facing second surface 112. The first sterile surface 110 of the second material ply 108 is removable from the sterile second surface 106 of the first material ply 102. The two material plies 102, 108 are coupled together with a mechanism such as, for example, two-sided tape 152 (See FIGS. 2 and 7).

The first surface 104 of the first material ply 102 is contaminated when it is placed against the equipment 122 or the non-sterile surface of a patient 124. The sterile second surface 106 of the first material ply 102 is sterile and, when application is complete and the second material ply 108 is removed, the sterile second surface 106 of the first material ply 102 will present a sterile surface to the sterile personnel.

The second material ply 108 is used during application of the sterile drape 100 as a barrier between the sterile second surface 106 of the first material ply 102 and the non-sterile personnel who apply the drape 100. Both surfaces 110, 112 of the second material ply 108 are sterile when the drape is removed from its packaging, but the second surface 112 of the second material ply 108 is allowed to be contaminated if a non-sterile person touches the drape during its application and positioning. The two plies 102, 108 are coupled together, for example by adhesive, thread, tape 152 (See FIG. 2), or a combination of any such devices, at specific points, along lines or at surfaces, so that the two drape plies 102, 108 behave as a single sheet during application. After application of the double ply sterile drape 100 to the patient 124 or equipment 122, the non-sterile person applying the drape may put their hands in a cavity 208, for example a glove-shaped cavity 210 defined in the second material ply 108 or outer material ply 204 and manipulate elements, for example a sterile device 142 of the first material ply 102 or inner material ply 202 so that these elements are properly configured or positioned.

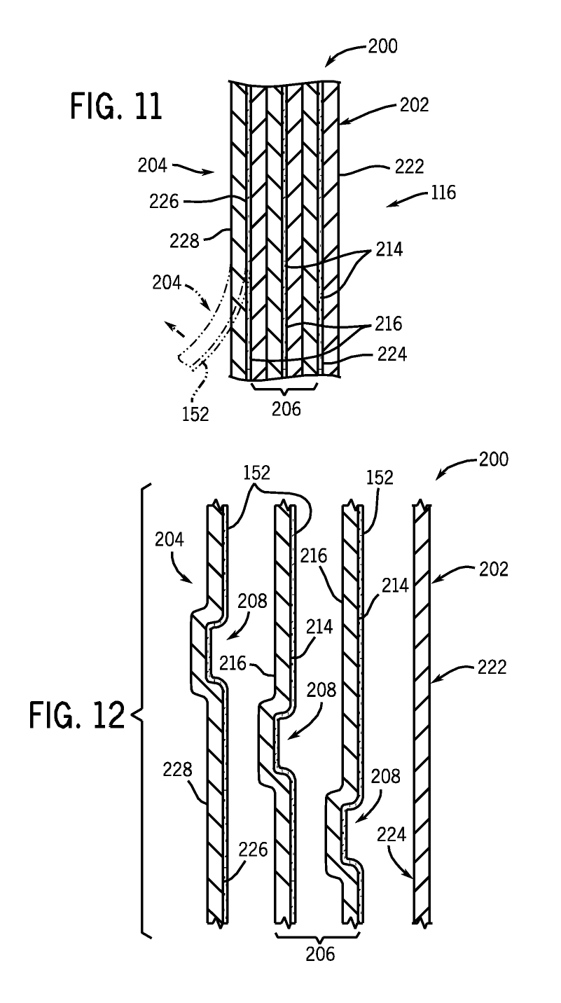

In a multi-ply drape 200 embodiment the innermost material ply 202 and outermost material ply 204 function as described above. For the plies of the plurality of material plies 206 each ply has a first sterile surface 214 and a second sterile surface 216. The sterile second surface 216 of the ply adjacent to the outermost ply is the sterile surface exposed when the outermost ply is removed. That ply 206 then becomes the new outermost ply and presents a sterile surface. When an outermost ply becomes contaminated it can be removed, exposing the next adjacent sterile ply, and this step can be repeated as necessary until the inner material ply 202 is exposed. Any number of plies can make up the multi-ply drape 200 (See FIGS. 11 and 12).

The outermost material ply 204 is used during application of the sterile drape 200 as a barrier between the sterile second surface 216 of the adjacent material ply and the non-sterile personnel who apply the drape 200. Both surfaces 226, 228 of the outer material ply 204 are sterile when the drape is removed from its packaging, but the second surface 228 of the outermost material ply 204 may be contaminated if a non-sterile person touches the drape during its application and positioning. The two plies 102, 108 of a dual-ply drape 100 or the multiple plies 202, 204, and 206 of a multi-ply drape 200 are coupled together, for example by adhesive, thread, tape 152 (See FIGS. 2, 11, and 12), or a combination of any such devices, at specific points, along lines or at surfaces, so that the two drape plies 102, 108 of a dual-ply drape or the multiple plies 202, 204, and 206 of a multi-ply drape 200 behave as a single sheet during application. After application of the multi-ply sterile drape 200 to the patient 124 or equipment 122, the non-sterile person applying the drape may put their hands in cavities, for example glove-shaped cavities 210 of the outer material ply 204 and manipulate the drape 200 so that those elements are properly configured.

The elements of a material ply needing manipulation may also be a framework 212 coupled to the first material ply 102 or innermost material ply 202 that facilitates shaping or positioning the first or innermost material ply in a desired shape or on a specific portion of an object 120. Examples of a specific portion of an object 120 are a limb of a patient 124, an eye piece of a microscope 130, and a monitor of an instrument. The framework may be a woven lattice incorporated in the material ply or it may be a series of members coupled together, to allow articulation, imbedded in the material ply. In another embodiment, the framework 212 is a network of gas filled tubes. The network can be inflated and deflated as determined by the user. The gas is air, or nitrogen or other suitable gas for use in a medical environment. In another embodiment the material ply may include a weight to position the material ply in a specific position on the object being draped. In another embodiment the framework 212 may be a slat coupled to an edge of the material ply and configured to press the material ply against another object, such as the inner wall of a bore of an MRI scanner or CT scanner (See FIG. 9).

After application of the double ply sterile drape 100 to the patient 124 or equipment 122 and manipulation of the elements of the first material ply 102, the second material ply 108 is removed from the first material ply 102 so that the sterile second surface 106 of the first material ply 102 is not contaminated. This leaves the sterile second surface 106 of the first material ply 102 exposed and sterile and the equipment 122 or patient 124 and operating table 128 successfully draped.

Initially the sterile drape 100, 200 is in a sterile package, for example, a box, bag, clear wrap or similar material. A user opens the package, removes the sterile drape 100, 200 and partially unfolds the sterile drape 100, 200. Such operations cause, usually, the outer surface 112, 228 (See FIGS. 2 and 11) of the outer material ply 104, 204 to become contaminated, i.e. non-sterile. The user then places the sterile drape 100, 200 on the object 118, and in some cases removably couples the drape 100, 200 to a portion 120 of the object 118. In some configurations, the first surface 104, 222 of the inner material ply 102, 202 will include an adhesive that will secure the ply 102, 202 to the object 118, but allow removal of the ply as determined by the user, i.e. medical person.

In some embodiments a sterile device 142, for example, suction tubes, cautery instruments, supplies, such as gauze, needles, sponges, instruments, wires 146, wire terminal 144, wire connector 148, RF coils or similar devices, used during a medical procedure is sandwiched between the first and second material ply 102, 108. (See FIGS. 1, 7, 8, and 12) In such case, the sterile device 142 is in contact with each of the sterile surfaces of the plies, with one ply configured to release from the sterile device 142.

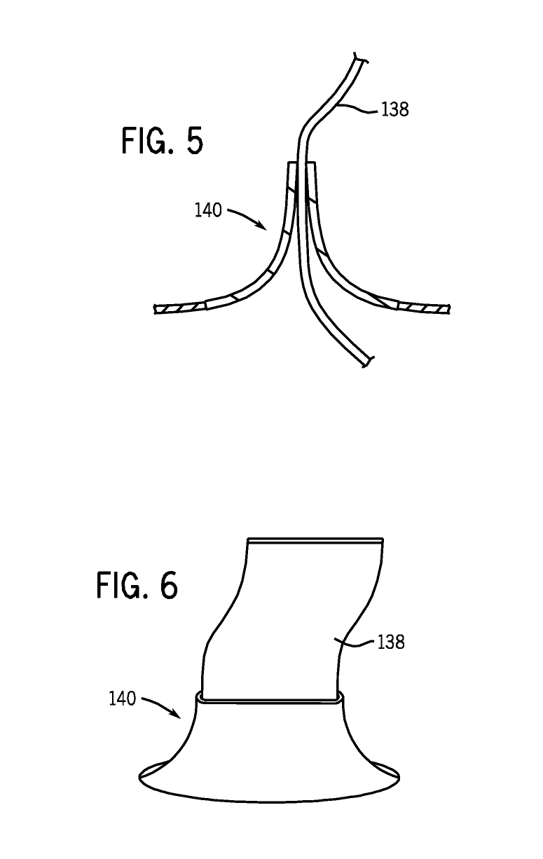

While applying the sterile drape 100, 200 to an object 118, the user can simply lay the sterile drape 100, 200 on the object 118. In some circumstances, the user can cover the object 118 with the sterile drape 100, 200 and secure the drape with a tensioning system 134. The tensioning system 134 includes a one-way ratchet mechanism 140 and a drawstring 138. The drawstring 138 can be configured as a cord or a ribbon (See FIGS. 5 and 6). In one embodiment the tensioning system 134 is disposed proximate an outer edge 136 of the sterile drape 100. In another embodiment the tensioning system 134 is within the perimeter 150 of the sterile drape 100 to secure the drape to equipment 122 (FIG. 3).

The one-way ratchet mechanism 140 is configured, for example to prevent the drawstring 138 from moving backward, i.e. opposite direction from the initial direction of movement. Such configuration also prevents contamination of the sterile surfaces 106, 110 of the two plies 102, 108 as the tensioning system 134 is operated. In a preferred embodiment, a one-way ratchet mechanism 140 is defined in each material ply (102, 108) [See FIG. 3]. It should be understood that a similar mechanism 140 may be included in the multi-ply sterile drape 200.

In another embodiment, the sterile drape 200 will include a plurality of material plies 206 disposed between an inner material ply 202 and an outer material ply 204. In such embodiment the outer most material ply can be removed, in succession, as the need arises, as determined by the user. Each ply of the plurality of material plies 206 includes a sterile first surface 214 and opposite facing second surface 216, so that as the outermost (last ply) ply is removed, a sterile field remains.

In another embodiment, each material ply is tinted a different color 218 to facilitate identification of the plies. A specific color is determined by the manufacturer or user of the multi-ply sterile drape 200. In another embodiment, at least one material ply is transparent 220, for example the outer material ply 204.

For purposes of this disclosure the phrase “proximal surface” means the surface of a ply closest to operating room personnel when they are applying the drape and the phrase “distal surface” means the surface of a ply farthest from the operating room personnel when they are applying the drape.

While the current application recites particular combinations of features in the claims appended hereto, various embodiments of the invention relate to any combination of any of the features described herein whether or not such combination is currently claimed, and any such combination of features may be claimed in this or future applications. Any of the features, elements, or components of any of the exemplary embodiments discussed above may be claimed alone or in combination with any of the features, elements, or components of any of the other embodiments discussed above.

For purposes of this disclosure, the term “coupled” means the mechanical joining of two components directly or indirectly to one another. Such joining may be stationary in nature or moveable in nature. Such joining may be achieved with the two components and any additional intermediate members being integrally formed as a single unitary body with one another or the two components and any additional member being attached to one another. Such adjoining may be permanent in nature or alternatively be removable or releasable in nature.

Although the foregoing description of the present mechanism has been shown and described with reference to particular embodiments and applications thereof, it has been presented for purposes of illustration and description and is not intended to be exhaustive or to limit the disclosure to the particular embodiments and applications disclosed. It will be apparent to those having ordinary skill in the art that a number of changes, modifications, variations, or alterations to the mechanism as described herein may be made, none of which depart from the spirit or scope of the present disclosure. The particular embodiments and applications were chosen and described to provide the best illustration of the principles of the mechanism and its practical application to thereby enable one of ordinary skill in the art to utilize the disclosure in various embodiments and with various modifications as are suited to the particular use contemplated. All such changes, modifications, variations, and alterations should therefore be seen as being within the scope of the present disclosure as determined by the appended claims when interpreted in accordance with the breadth to which they are fairly, legally, and equitably entitled.

What is claimed is:

1. A sterile drape comprising:
an inner material ply having a first surface and an opposite facing sterile second surface;
an outer material ply having a first sterile surface and an opposite facing second surface; and
a plurality of material plies disposed between the inner material ply and the outer material ply, with each ply of the plurality of material plies including a first sterile surface and an opposite facing second sterile surface, with the first sterile surface of each material ply removably coupled to the sterile second surface of the adjacent material ply.

2. The sterile drape of claim 1, configured in one of a tube, a planar sheet, and a predetermined shape.

3. The sterile drape of claim 1, wherein the inner material ply, the outer material ply, and each of the plurality of material plies are removably coupled together with one of adhesive, tape, and thread.

4. The sterile drape of claim 1, further comprising a sterile device attached between at least two of the sterile surfaces of any two adjacent material plies configured to release from the sterile device.

5. The sterile drape of claim 1, further comprising a tensioning system configured to enclose a portion of an object with one of the inner material ply, the plurality of material plies and the outer material ply.

6. The sterile drape of claim 5, with the tensioning system including a drawstring disposed proximate one of within the perimeter of the sterile drape and an outer edge of one of the inner material ply, the plurality of material plies and the outer material ply, with the drawstring extending through a one-way ratchet mechanism defined in at least one of the plies.

7. The sterile drape of claim 1, wherein each material ply have characteristics of resistance to blood, aqueous fluid, abrasion, and are lint free.

8. The sterile drape of claim 1, further comprising a wire terminal configured to receive a wire connector on each of the first and second surfaces of at least one of the inner material ply, the outer material ply and each material ply in the plurality of material plies, with the wire terminal coupled to the wire terminal in the adjacent material ply to provide a continuous electrical connection through the drape.

9. The sterile drape of claim 1, further comprising a cavity defined in at least the outer material ply, with the cavity defined as a glove.

10. The sterile drape of claim 1, further comprising the inner material ply is colored and the outer material ply is transparent.

11. The sterile drape of claim 1 further comprising a framework coupled to the inner material ply and configured to be manipulated to a desired shape.

12. A method to provide a sterile field on an object, the method comprising:
obtaining a sterile drape package;
opening the package and removing the sterile drape, wherein the sterile drape is configured in one of a tube, a planar sheet, and a predetermined shape;
unfolding the sterile drape to cover selected parts of the object, with the sterile drape comprising:
an inner material ply having a first surface and an opposite facing sterile second surface;
an outer material ply having a first sterile surface and an opposite facing second surface; and
a plurality of material plies disposed between the inner material ply and the outer material ply, with each ply of the plurality of material plies including a first sterile surface and an opposite facing second sterile surface, with the first sterile surface of each material ply removably coupled to the sterile second surface of the adjacent material ply, with one of adhesive, tape, and thread; and
removing the outer material ply from an adjacent material ply exposing the sterile second surface of the adjacent material ply without contaminating the sterile second surface of the adjacent material ply, the sterile second surface of the adjacent material ply providing the sterile field.

13. The method to provide a sterile field on an object of claim 12, wherein the sterile drape includes a framework in at least one material ply, the method further includes manipulating the framework in the material ply and conforming the sterile drape to a desired shape.

14. The method to provide a sterile field on an object of claim 12, with the sterile drape further comprising a tensioning system, the method including operating the tensioning system to constrict a portion of the sterile drape around the object and selectively position the sterile drape.

15. The method to provide a sterile field on an object of claim 14, with the tensioning system including a drawstring disposed proximate an outer edge of one of the inner material ply, the plurality of material plies and the outer material ply, with the drawstring extending through a one-way ratchet mechanism defined in one of the plies.

16. The method to provide a sterile field on an object of claim 12, further comprising using a sterile device attached to each of the sterile surfaces of adjacent material plies, with at least one of the adjacent material plies configured to release from the sterile device.

17. The method to provide a sterile field on an object of claim 12, wherein the object being draped is one of equipment and a patient.

18. The method to provide a sterile field on an object of claim 13, wherein the patient is a human.

19. The method to provide a sterile field on an object of claim 12, further comprising using a wire terminal configured to receive a wire connector on each of the first and second surfaces of at least one of the inner material ply, the outer material ply and each material ply in a plurality of material plies, with the wire terminal coupled to the wire terminal in the adjacent material ply to provide a continuous electrical connection through the drape.

20. The method to provide a sterile field on an object of claim 12, wherein each material ply have characteristics of resistant to blood, aqueous fluid, abrasion, and are lint free.

21. A sterile drape comprising:
a first material ply having a first surface and an opposite facing sterile second surface; and
a second material ply having a first sterile surface and an opposite facing second surface, with the first sterile surface of the second material ply removably coupled to the sterile second surface of the first material ply.

22. The sterile drape of claim 21, configured in one of a tube and a planar sheet.

23. The sterile drape of claim 21, wherein the first material ply and the second material ply are removably coupled together with one of adhesive, tape, and thread.

24. The sterile drape of claim 21, further comprising a sterile device attached to each of the sterile surfaces of the first and second material ply, with at least one of the first and second material ply configured to release from the sterile device.

25. The sterile drape of claim 21, further comprising a tensioning system configured to enclose a portion of an object with one of the first material ply and second material ply.

26. The sterile drape of claim 25, with the tensioning system including a drawstring disposed proximate one of within the perimeter of the sterile drape and an outer edge of one of the first material ply and the second material ply, with the drawstring extending through a one-way ratchet mechanism defined in one of the plies.

27. The sterile drape of claim 21, wherein the first and second material ply have characteristics of resistance to blood, aqueous fluid, abrasion, and are lint free.

28. The sterile drape of claim 21, further comprising a wire terminal configured to receive a wire connector on each of the first and second surfaces of one of the first material ply and second material ply and provide a continuous electrical connection through the drape.
